(19) United States
(12) Patent Application Publication
(10) Pub. No.: US 2015/0151058 A1
(43) Pub. Date: Jun. 4, 2015

(54) NEBULIZER APPARATUS COMPRISING A MESH

(71) Applicant: FLAEM NUOVA S.P.A., Desenzano del Garda (Brescia) (IT)

(72) Inventors: Luigi Abate, Desenzano del Garda (Brescia) (IT); Ricardo Abate, Desenzano del Garda (Brescia) (IT); Nicola Fraccaroli, Desenzano del Garda (Brescia) (IT)

(73) Assignee: FLAEM NUOVA S.P.A., Desenzano del Garda (Brescia) (IT)

(21) Appl. No.: 14/412,880

(22) PCT Filed: Jul. 4, 2013
(86) PCT No.: PCT/EP2013/001964

§ 371 Date: Jan. 5, 2015

(30) Foreign Application Priority Data
Jul. 6, 2012 (IT) MI2012A 001194

(51) Int. Cl. A61M 11/00; B29C 45/14; A61M 15/00

(52) U.S. Cl. CPC A61M 11/005; A61M 11/003; A61M 15/0021; B29C 45/14311; B29C 45/14336; A61M 2207/10; B29K 2083/005

(57) ABSTRACT

Mesh (1) for nebulizer apparatus for medical applications, with a structure which is particularly rigid and easy to fix in position inside the nebulizer apparatus, comprising a vibratile foil (10) crossed by a plurality of nebulizing holes (11) and a peripheral embossed edge (12) integral with the vibratile foil (10).

### FIELD OF APPLICATION

The present invention relates to a so-called mesh for a nebulizer apparatus for medical applications, a nebulizer apparatus comprising said mesh, and a corresponding method for manufacturing the mesh.

The aforementioned mesh is usefully employed in the medical sector, in particular in the sector of aerosol therapy.

### Prior Art

The administration of drugs aerially by means of special nebulization devices is a therapeutic practice normally used in connection with many diseases, in particular those affecting the respiratory apparatus.

Nebulization allows in particular a drug in liquid form to be divided up into a plurality of very fine particles, having a diameter of a few micrometers which may be easily inhaled by a patient.

In addition to conventional compressed-air nebulizers, so-called ultrasonic nebulizers have also been developed with time, these being lighter and more silent and making use of the high-frequency vibration of a piezoelectric component in order to atomize the liquid drug contained in a reservoir.

Recently ultrasonic nebulizers have been further improved with the introduction of what is known as “vibrating mesh technology”. Instead of providing a piezoelectric component on the bottom of the nebulizer reservoir, this new technology envisages the insertion in a dispensing outlet of a very thin membrane provided with a multitude of micrometric holes. By causing the membrane to vibrate at high frequency, very fine nebulization of the drug through the holes is obtained, without, however, encountering the problems of hyperthermia and excessive dispersion of the drug which constitute the limitations of conventional ultrasonic nebulizers.

The vibratile membrane which allows nebulization, referred to in the sector by the term “mesh”, is a metallic foil with a thickness of a few micrometers (typically 50 μm). It has an extremely large number of holes (typically between 1000 and 7000) with a diameter of a few micrometers. The holes may be produced with a laser or formed directly in the foil made by means of electroforming.

While fully meeting the needs of the sector, nebulizers with a vibratile mesh nevertheless pose a number of problems.

Because of its micrometric thickness, in fact, the mesh element is very fragile. Extreme care is thus required during assembly and maintenance of the device in order to avoid structural damage to this element.

The element is also difficult to assemble inside a mechanical assembly or combine with other structural elements. In view of its small thickness, the mesh must in fact necessarily be fixed against a shoulder by means of a fixing ring. This results in a long and delicate procedure needed to assemble it, as well as a relatively complex structure of the nebulizer.

The technical problem forming the basis of the present invention is therefore that of devising a mesh which solves the problems described in connection with the prior art and which in particular is sufficiently strong and can be easily inserted into the structure of a nebulizer device.

### SUMMARY OF THE INVENTION

The aforementioned technical problem is solved by a mesh for nebulizer apparatus for medical applications, comprising a vibratile foil crossed by a plurality of nebulizing holes and a peripheral embossed edge integral with the vibratile foil. An overmolded gasket is also provided on top of said peripheral embossed edge.

The peripheral embossed edge advantageously helps strengthen the mesh, improving its mechanical properties and facilitating incorporation of the mesh in other parts of the internal structure of a nebulizer apparatus.

For instance, the mesh may be easily incorporated or enclosed within the above mentioned overmolded gasket.

The overmolded gasket may be made of an elastomer, such as a silicone rubber—preferably a bicomponent silicone rubber—or a thermoplastic elastomer (TPE). The gasket may be easily manufactured by injection molding of these materials within a mold in which the peripheral embossed edge is set.

It should be noted that the qualifying term “peripheral” attributed to the edge of the mesh is to be understood with reference to the perforated foil which forms the functional part of the mesh; alternative embodiments comprising further structures or foils which extend beyond the peripheral edge are therefore not excluded.

It should be noted moreover that the edge of the mesh must not necessarily extend continuously along the entire periphery of the perforated foil, it being possible to envisage one or more interruptions along the extension thereof. These interruptions may also be partial, namely cause thinning of the edge without, however, being reduced to the thickness of the foil. In other words, it is possible to envisage a non-uniform thickness of the edge of the mesh.

The vibratile foil has a uniform thickness preferably smaller than 0.1 mm, for example of 0.05 mm; the peripheral edge has preferably a maximum thickness greater than 0.2 mm, for example 0.5 mm.

The nebulizing holes, which may have a cylindrical form or any other form known in the art, preferably have a size smaller than 10 μm. An ideal size is, for example, 5 μm.

The peripheral edge may have an annular profile, for example with a semicircular cross-section (but sections with a different shape, for example rectangular, triangular, and other shapes, are also possible). It may be separated from a perforated central portion of the vibratile foil by a continuous rim without holes.

The mesh may have a flat surface, while the opposite surface has the embossed edge.

In order to allow better structural integration of the two elements, the vibratile foil and the peripheral embossed edge may be made of the same material, preferably metallic material, for example nickel.

The aforementioned technical problem is also solved by a nebulizer apparatus for medical applications, inside which a fluid path intercepted by a mesh of the type indicated above is defined, said fluid path connecting a reservoir for containing a drug in liquid form to a dispensing mouth, said nebulizer apparatus also comprising an oscillating element predisposed for causing oscillation of the vibratile foil of said mesh.

The overmolded gasket thus performs the two-fold function of keeping the vibratile foil in elastic contact with the oscillating element and of sealing the reservoir in order to avoid liquid spillage.

The abovementioned technical problem is also solved by a method for manufacturing a mesh for a nebulizer apparatus for medical applications, comprising: a step of manufacturing a vibratile foil, crossed by a plurality of nebulizing holes; and a subsequent step of selectively depositing material on the periphery of said vibratile foil, so as to realize a peripheral embossed edge integral with the vibratile foil.

With this method it is advantageously possible to obtain a mesh with an improved structure of the type described above.

The vibratile foil may be advantageously obtained by means of electroforming. This process may for example envisage depositing nickel on top of a preformed matrix.

The deposition of material on the periphery of the vibratile foil may be advantageously obtained by means of a photolithographic process, optionally in combination with a further electroforming process.

In the case of a vibratile foil made of nickel, for example, it is possible to envisage the application of a photoresist on one of the sides of this foil, then the selective removal of the photoresist from only peripheral edge and finally a further electroforming step, where the nickel adheres only on this edge from which the photoresist has been removed.

The vibratile foil and the peripheral embossed edge may be both made of a first metallic material (for example nickel), the method also comprising a step of depositing a layer of a second biocompatible material (for example gold) on top of the vibratile foil.

Deposition of said second material, which may be performed again by means of electroforming, allows the mesh to be rendered biocompatible and the size of the nebulizing holes to be calibrated.

The layer of second biocompatible material deposited may be between 1 μm and 5 μm, and preferably equal to 3 μm.

The method also advantageously comprises a step of overmolding a gasket (for example made of rubber, silicone or other elastomer, for instance a thermoplastic elastomer) on top of the peripheral embossed edge.

It should be noted that easy overmolding of a gasket is advantageously permitted by the presence of the peripheral embossed edge.

In overmolding the gasket, the vibratile foil may be placed within a mold wherein an elastomer is subsequently injected in liquid form, the elastomer being subsequently solidified so as to form the gasket enclosing the peripheral embossed edge.

In a preferred embodiment, the solidification of the elastomer is obtained by heat curing; in the preferred case of a liquid bicomponent silicone rubber, the process is performed by heating the mold at a temperature of approximately 160° C. for at least one minute.

Before overmolding of the gasket, the peripheral embossed edge of the mesh may be covered with a primer that promotes adhesion between the elastomer and the peripheral embossed edge. Otherwise, an elastomer such as a silicone rubber would not adhere on the metallic coating (for instance gold) of the mesh.

Further characteristic features and advantages will appear more clearly from the detailed description, provided hereinbelow, of a preferred, but not exclusive embodiment of the present invention, with reference to the attached figures provided by way of a non-limiting example.

### BRIEF DESCRIPTION OF THE DRAWINGS

FIG. 1 shows a cross-sectional side of the mesh according to the present invention;

FIG. 2 shows an enlarged detail of the mesh shown in FIG. 1;

FIG. 3 shows a cross-sectional side view of the mesh shown in FIG. 1 with an overmolded gasket;

FIG. 4 shows a cross-sectional side view of a nebulizer apparatus according to the present invention, comprising the mesh shown in FIG. 1;

FIG. 5 shows a view of a detail of the nebulizer apparatus shown in FIG. 4 in an open configuration.

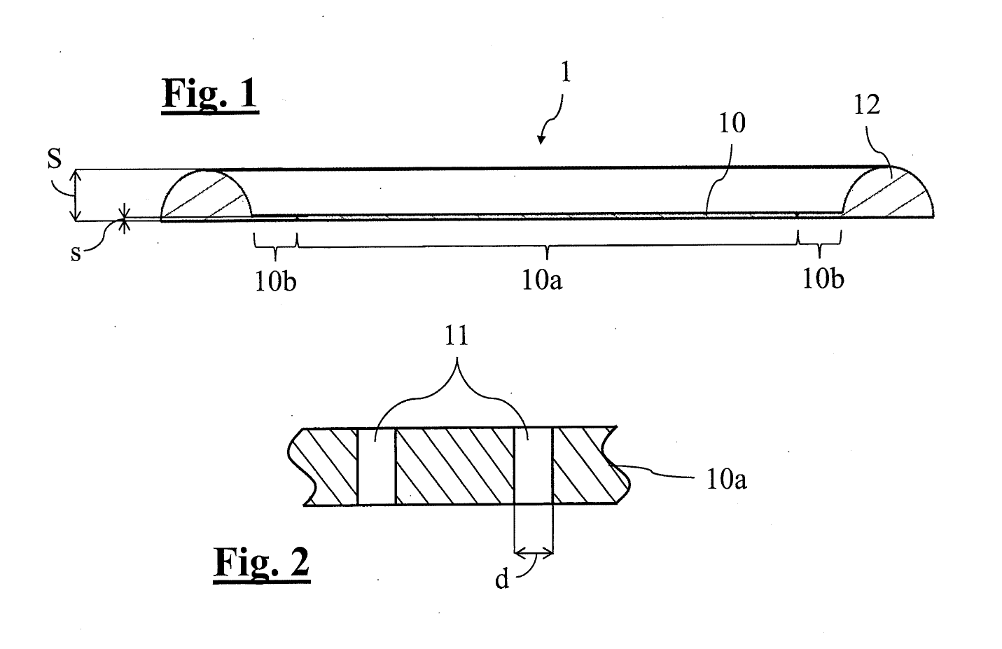

### DETAILED DESCRIPTION

With reference to the accompanying figures, 1 denotes in general a mesh which can be assembled inside a nebulizer apparatus 100 for medical applications, in particular for aerosol therapy.

The mesh 1 takes the form of a circular element comprising a vibratile foil 10 surrounded by a peripheral embossed edge 12.

The vibratile foil 10 is made of metallic material, preferably nickel coated with gold, according to a process described below.

The vibratile foil 10 has a uniform thickness s smaller than a tenth of a millimeter. In particular, the uniform thickness s is equal to 50 μm. Given its thickness, the vibratile foil 10 may be made to oscillate by a special component of the nebulizer apparatus.

The vibratile foil 10 is crossed by a plurality of nebulizing holes 11 which allow nebulization of a medical liquid to be dispensed. The nebulizing holes 11 have a size or a diameter d smaller than 10 μm; in particular, they have a diameter d, for example, of 5 μm. It should be noted that the cylindrical form of the nebulizing holes 11 shown in FIG. 2 is to be understood as being purely exemplary in nature.

The nebulizing holes 11 are formed in a central area 10a of the vibratile foil 10; a continuous rim 10b separates this central area from the peripheral embossed edge 12.

The peripheral embossed edge 12 is integral with the vibratile foil 10. This edge helps strengthen the vibratile foil 10 and defines moreover a fastening structure which allows easy integration of the part with further components (for example, as described below, allows overmolding of an elastomer gasket).

In the specific embodiment shown here, the peripheral edge 12 has an annular profile with a semi-circular cross-section. The mesh 1 thus has a continuous surface and an opposite surface from which the peripheral embossed edge 12 protrudes.

Purely by way of example, the dimensions of a specific embodiment of the mesh 1 according to the present invention are given. The overall diameter of the mesh is equal to 8.5 mm: the central area 10a has a diameter of 5.5 mm; the continuous rim 10b around the latter has a radial extension of 0.5 mm; the peripheral embossed edge 12 has a radial extension of 1 mm. The maximum thickness S of the peripheral embossed edge is 0.5 mm.

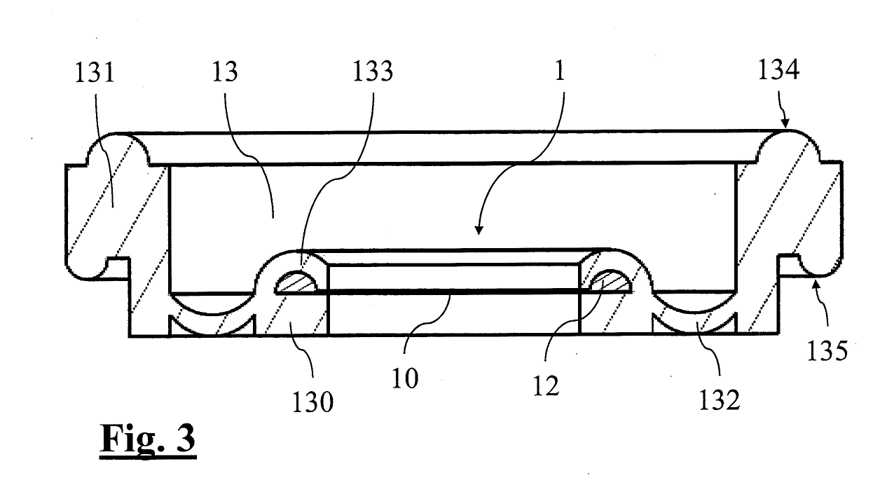

The mesh 1 may comprise an overmolded gasket 13 made of a single material: preferably rubber, such as a thermoplastic rubber (TPE) a silicone rubber, or the like. This gasket is shown by way of example in FIG. 3.

The gasket 13 comprises an inner collar 130, intended to retain the mesh 1, and an outer collar 131, intended to be fixed onto the nebulizer apparatus 100.

The two collars are connected by a convex connecting flange 132. In particular, when the gasket 13 is mounted on the nebulizer apparatus 100, the flange 132 has its convexity directed upstream and its concavity downstream, with reference to the flow of the fluid supplied through the apparatus.

The inner collar 130 comprises a cylindrical ring arranged upstream (still with reference to the assembled configuration of the mesh 1 together with the gasket 13) against which the flat surface of the mesh 1 described above bears. In order to keep this mesh 1 in position, the inner collar 130 comprises a fixing lip 133. The fixing lip 133 extends from the outer periphery of the surface downstream of the aforementioned cylindrical ring and is arranged entirely over the mesh 1 so as to be closed against the inner periphery of the surface of the cylindrical ring.

The outer collar 131 extends in the manner of a cylindrical sleeve, extending in a downstream direction. At its downstream end, the cylindrical sleeve defines a first mating surface 134, characterized by the presence of a circumferential relief with a semi-circular cross-section. A second mating surface 135 situated opposite the first surface, with a similar circumferential relief having a semi-circular cross-section, is defined on an intermediate outer shoulder of the cylindrical sleeve.

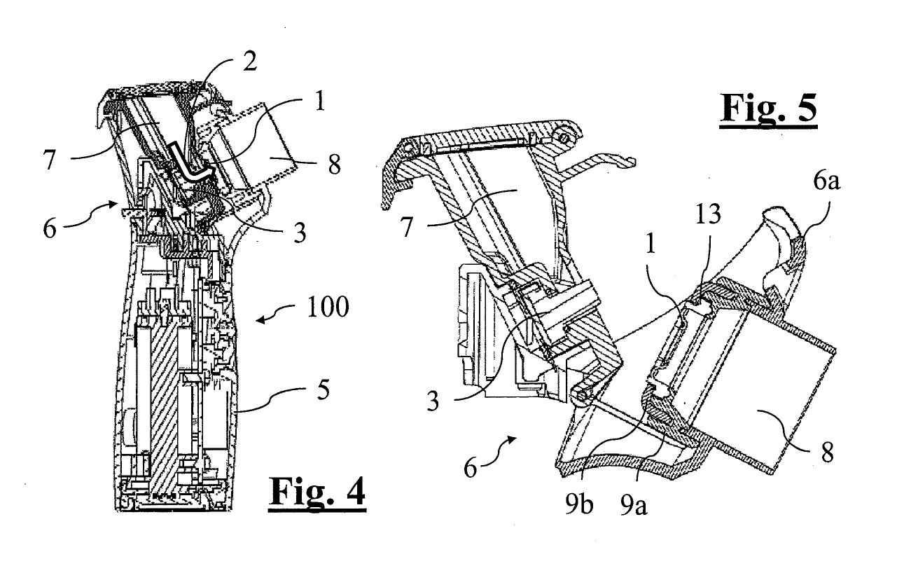

The nebulizer apparatus 100, which is visible in its entirety in FIG. 4, is described here in brief, with identification in particular of the parts which cooperate directly with the mesh 1 discussed above.

The nebulizer apparatus comprises a gripping part 5 above which a dispensing assembly 6 of the device is fixed. This dispensing assembly 6 comprises a reservoir 7 for containing a drug in liquid form which is accessible at the top by means of a special re-closable flap. The dispensing assembly further comprises a dispensing mouth 8 for the nebulized liquid.

A fluid path 2 is defined between the reservoir 7 and the dispensing mouth 8 and passes through the mesh 1 arranged at the inlet of the dispensing mouth. An oscillating element 3 is arranged upstream of said mesh 1, one end thereof being arranged in contact with the mesh 1 and being predisposed to cause oscillation of the vibratile foil 10. The oscillating element 3 is suitably connected to control electronics for receiving command signals, accessible on the gripping part 5.

The mesh 1, which is provided with the overmolded gasket 13, may be easily mounted in its position along the fluid path 2. In fact, as can be seen in FIG. 5, the dispensing portion 6 comprises a hinged portion 6a, opening of which allows access to the upstream end of the dispensing mouth 8.

A first fastening ring 9a and a second fastening ring 9b are associated with this end of the dispensing mouth 8. The first fastening ring 9a engages directly on the end of the dispensing mouth, while the second fastening ring 9b engages on the free end of the first fastening ring 9a. The gasket 13 of the mesh 1 is gripped between the first fastening ring 9a and the second fastening ring 9b; in particular, the first mating surface 134 mates with a matching surface of the first fastening ring 9a, while the second mating surface 135 mates with a matching surface of the second fastening ring 9b.

The gasket 13 has a two-fold function. On one hand, it keeps the vibratile foil 10 of the mesh 1 in elastic contact with the oscillating element 3. On the other hand, it performs a sealing function preventing spillage from the reservoir 7.

The mesh 1 according to the present invention is formed by means of the method described below.

Firstly, the vibratile foil 10 with its nebulizing holes 11 is formed by means of electroforming. The electroforming process envisages the deposition, by means of electrolysis, of a layer of nickel on a matrix which defines the desired form for the mesh, with many holes. The deposited layer has a thickness of about 0.05 mm.

The peripheral embossed edge 12 is then formed on top of the foil 10 obtained by means of electroforming. This edge may be obtained in particular by means of the photolithographic technique, combined with the aforementioned electroforming technique.

For example it is possible to coat the foil 10 obtained beforehand with a photoresist, which is then selectively removed from the peripheral edge alone. During a following electroforming step, the nickel is deposited only on the peripheral edge not covered by the photoresist, defining the desired relief (with a maximum thickness of about 0.5 mm).

A subsequent step of the method envisages a further deposition of gold onto the structure obtained. The layer of gold, which is for example between 1 μm and 5 μm (preferably 3 μm), makes the mesh 1 biocompatible and calibrates the outlet holes to the desired diameter, for example 5 μm.

The mesh 1 thus obtained may then be overmolded with rubber or silicone so as to obtain the gasket 13 described above.

The overmolding process is obtained by injection molding of a bicomponent silicone rubber.

Beforehand, the peripheral edge of the foil 10 is covered with a suitable primer that promotes the adhesion between the silicone rubber and the gold coating of the foil.

The foil 10 treated with the primer is then inserted into a mold, which is shaped in the form of the gasket to be manufactured. The liquid silicone is then pressurized and injected therein by means of a suitable injector device. Within the mold, the silicone is heat cured by raising its temperature to about 160° C. for at least one minute.

The heat cured silicone, which has taken its final gasket 13 form as defined by the inner shape of the mold, is then extracted from the mold itself and is ready for insertion within the nebulizer apparatus 100.

The mold is made of a metallic material, usually steel. Since the cured silicone does not adhere to metal, the final gasket 13 is easily extracted.

A thermoplastic elastomer (TPE) may be employed as an alternative to silicone rubber.

1. A mesh for nebulizer apparatus for medical applications, comprising a vibratile foil crossed by a plurality of nebulizing holes, further comprising a peripheral embossed edge integral with the vibratile foil, and an overmolded gasket on top of said peripheral embossed edge.

2. The mesh according to claim 1, wherein said overmolded gasket encloses said peripheral embossed edge.

3. The mesh according to claim 1, wherein the overmolded gasket is made of an elastomer.

4. The mesh according to claim 1, wherein said vibratile foil has a uniform thickness below 0.1 mm, the peripheral edge having a maximum thickness above 0.2 mm.

5. The mesh according to claim 1, wherein said nebulizing holes have a dimension below 10 μm.

6. The mesh according to claim 1, wherein the peripheral edge has a continuous annular profile.

7. The mesh according to claim 1, wherein the peripheral edge has a discontinuous annular profile, interrupted by one or more sections having a lower thickness with respect to a maximum thickness of the vibratile foil.

8. The mesh according to claim 7, wherein at least one of said sections has a thickness equal to that of the vibratile foil.

9. A nebulizer apparatus for medical applications, wherein a fluid path intercepted by a mesh according to claim 1 is defined, said fluid path connecting a reservoir for containing a drug in liquid form to a dispensing mouth, said nebulizer apparatus further comprising an oscillating element predisposed for oscillating the vibratile foil of said mesh, said overmolded gasket performing the two-fold function of keeping the vibratile foil in elastic contact with the oscillating element and of sealing the reservoir in order to avoid liquid spillage.

10. A method for manufacturing a mesh for nebulizer apparatus for medical applications, comprising: a step of manufacturing a vibratile foil crossed by a plurality of nebulizing holes; a subsequent step of selectively deposing a material on the periphery of said vibratile foil, so as to realize a peripheral embossed edge integral with the vibratile foil, and a subsequent step of overmolding a gasket on top of the peripheral embossed edge.

11. The method for manufacturing a mesh according to claim 10, wherein, in overmolding the gasket, the vibratile foil is placed within a mold wherein an elastomer is subsequently injected in liquid form, the elastomer being subsequently solidified so as to form the gasket enclosing the peripheral embossed edge.

12. The method for manufacturing a mesh according to claim 11, wherein, before overmolding of the gasket, the peripheral embossed edge of the mesh is covered with a primer that promotes adhesion between the elastomer and the peripheral embossed edge.

13. The method for manufacturing a mesh according to claim 10, wherein the step of deposing a material on the periphery of the vibratile foil is obtained through a photolithographic process.

14. The method for manufacturing a mesh according to claim 10, wherein said vibratile foil is obtained through electroforming.

15. The method for manufacturing a mesh according to claim 10, wherein said vibratile foil and said peripheral embossed edge are both realized in a first metallic material, the method further comprising a step of deposing a layer of a second biocompatible material on top of the vibratile foil.
